(19) United States
(12) Patent Application Publication
(10) Pub. No.: US 2024/0065799 A1
(43) Pub. Date: Feb. 29, 2024

(54) SYSTEMS AND METHODS FOR MEDICAL ASSISTANT

(71) Applicant: SHANGHAI UNITED IMAGING INTELLIGENCE CO., LTD., Shanghai (CN)

(72) Inventors: Terrence CHEN, Cambridge, MA (US); Ziyan WU, Cambridge, MA (US); Shanhui SUN, Cambridge, MA (US); Arun INNANJE, Cambridge, MA (US); Benjamin PLANCHE, Cambridge, MA (US); Abhishek SHARMA, Cambridge, MA (US); Meng ZHENG, Cambridge, MA (US)

(73) Assignee: SHANGHAI UNITED IMAGING INTELLIGENCE CO., LTD., Shanghai (CN)

(21) Appl. No.: 17/823,953

(22) Filed: Aug. 31, 2022

(51) Int. Cl. A61B 90/00; G06T 7/00; G06T 7/30; G06T 7/50; G06T 7/70

(52) U.S. Cl. CPC A61B 90/36; G06T 7/0012; G06T 7/30; G06T 7/50; G06T 7/70; A61B 2090/366; G06T 2207/30004

(57) ABSTRACT

The present disclosure provides systems and methods for medical assistant. The systems may obtain position information of a target inside a subject during an operation. The systems may also determine depth information of the target with respect to an operational region of the subject based on the position information. The systems may further direct an optical projection device to project an optical signal representing the depth information on a surface of the subject.

### TECHNICAL FIELD

The present disclosure generally relates to medical field, and more particularly, relates to systems and methods for medical assistant.

### BACKGROUND

In medical operations (e.g., a surgery), a user (e.g., a doctor) generally obtains relevant information associated with a target (e.g., a tumor) based on a scanned image (e.g., a CT image) of the subject acquired before the medical operation. In some cases, a guidance may be provided to the user during the medical operation based on a scanned image acquired during the medical treatment. However, not only it takes time to obtain the scanned image during the medical operation, but also the guidance generally can't be intuitively provided to the user. Therefore, it is desirable to provide systems and methods for medical assistant to improve the efficiency and convenience of the medical assistant.

### SUMMARY

In an aspect of the present disclosure, a method for medical assistant is provided. The method may be implemented on at least one computing device, each of which may include at least one processor and a storage device. The method may include obtaining position information of a target inside a subject during an operation. The method may include determining depth information of the target with respect to an operational region of the subject based on the position information. The method may further include directing an optical projection device to project an optical signal representing the depth information on a surface of the subject.

In some embodiments, the obtaining position information of a target inside a subject during an operation may include obtaining an optical image of the subject captured by a first acquisition device, obtaining a scanned image of the subject captured by a second acquisition device, and determining the position information of the target inside the subject during the operation based on the optical image and the scanned image. The scanned image may include the target.

In some embodiments, the determining the position information of the target inside the subject during the operation based on the optical image and the scanned image may include establishing a subject model corresponding to the subject based on the optical image, aligning the scanned image with the subject model, and determining the position information of the target inside the subject during the operation based on the aligned scanned image and the aligned subject model.

In some embodiments, the directing an optical projection device to project an optical signal representing the depth information on a surface of the subject may include determining a projection instruction based on the depth information and directing the optical projection device to project the optical signal representing the depth information on the surface of the subject based on the projection instruction. The projection instruction may be configured to direct a projection operation of the optical projection device.

In some embodiments, the determining a projection instruction based on the depth information may include obtaining an instruction generation model and determining the projection instruction based on the depth information and the instruction generation model.

In some embodiments, the determining a projection instruction based on the depth information may include determining updated depth information of the target with respect to the operational region of the subject based on at least one of updated position information of the target or position information of a surface level of the operational region and determining an updated projection instruction based on the updated depth information.

In some embodiments, the determining a projection instruction based on the depth information may include obtaining environment information associated with the subject and determining the projection instruction based on the environment information associated with the subject and the depth information.

In some embodiments, the projection instruction may be associated with signal information included in the optical signal. The signal information included in the optical signal may include at least one of color information of the optical signal or position information of the optical signal projected on the surface of the subject.

In some embodiments, the color information of the optical signal may indicate the depth information of the target with respect to the operational region.

In some embodiments, the color information of the optical signal may be associated with a type of the target.

In another aspect of the present disclosure, a system for medical assistant is provided. The system may include a controller and an optical projection device. The controller may be configured to obtain position information of a target inside a subject during an operation, determine depth information of the target with respect to an operational region of the subject based on the position information, and direct the optical projection device to project an optical signal representing the depth information on a surface of the subject. The optical projection device may be configured to project the optical signal representing the depth information on the surface of the subject.

In still another aspect of the present disclosure, a non-transitory computer-readable medium storing at least one set of instructions is provided. When executed by at least one processor, the at least one set of instructions may direct the at least one processor to perform a method. The method may include obtaining position information of a target inside a subject during an operation, determining depth information of the target with respect to an operational region of the subject based on the position information, and directing an optical projection device to project an optical signal representing the depth information on a surface of the subject.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary medical assistant system according to some embodiments of the present disclosure;

FIG. 2 is a block diagram illustrating an exemplary processing device according to some embodiments of the present disclosure;

FIG. 3 is a flowchart illustrating an exemplary process for medical assistant according to some embodiments of the present disclosure;

FIG. 4 is a schematic diagram illustrating an exemplary projection of an optical signal according to some embodiments of the present disclosure;

FIG. 5 is a flowchart illustrating an exemplary process for determining position information of a target inside a subject during an operation according to some embodiments of the present disclosure;

FIG. 6 is a schematic diagram illustrating an exemplary process for determining position information of a target inside a subject during an operation according to some embodiments of the present disclosure;

FIG. 7 is a schematic diagram illustrating an exemplary process for determining a projection instruction according to some embodiments of the present disclosure;

FIG. 8 is a flowchart illustrating an exemplary process for determining an updated projection instruction according to some embodiments of the present disclosure;

FIG. 9A is a schematic diagram illustrating an exemplary projection process during an operation according to some embodiments of the present disclosure;

FIGS. 9B-9D are schematic diagrams illustrating exemplary projections of an optical signal under different situations according to some embodiments of the present disclosure;

FIG. 10 is a flowchart illustrating an exemplary process for determining a projection instruction according to some embodiments of the present disclosure; and

FIG. 11 is a schematic diagram illustrating an exemplary projection of an optical signal according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

In the present disclosure, the subject may include a biological object and/or a non-biological object. The biological object may be a human being, an animal, a plant, or a specific portion, organ, and/or tissue thereof. For example, the subject may include the head, the neck, the thorax, the heart, the stomach, a blood vessel, a soft tissue, a tumor, a nodule, or the like, or any combination thereof. In some embodiments, the subject may be a man-made composition of organic and/or inorganic matters that are with or without life. The terms “object” and “subject” are used interchangeably in the present disclosure.

In the present disclosure, the term “image” may refer to a two-dimensional (2D) image, a three-dimensional (3D) image, or a four-dimensional (4D) image (e.g., a time series of 3D images). In some embodiments, the term “image” may refer to an image of a region (e.g., a region of interest (ROI)) of a subject. In some embodiment, the image may be a medical image, an optical image, etc.

In the present disclosure, a representation of an object (e.g., a subject, a patient, or a portion thereof) in an image may be referred to as “object” for brevity. For instance, a representation of an organ, tissue (e.g., a heart, a liver, a lung), or an ROI in an image may be referred to as the organ, tissue, or ROI, for brevity. Further, an image including a representation of an object, or a portion thereof, may be referred to as an image of the object, or a portion thereof, or an image including the object, or a portion thereof, for brevity. Still further, an operation performed on a representation of an object, or a portion thereof, in an image may be referred to as an operation performed on the object, or a portion thereof, for brevity. For instance, a segmentation of a portion of an image including a representation of an ROI from the image may be referred to as a segmentation of the ROI for brevity.

In the present disclosure, the terms “signal (signal portion)” and “operational region” are used interchangeably.

The present disclosure relates to systems and methods for medical assistant. The systems may obtain position information of a target inside a subject during an operation, and determine depth information of the target with respect to an operational region of the subject based on the position information. Further, the systems may direct an optical projection device to project an optical signal representing the depth information on a surface of the subject. Therefore, the user may obtain the depth information directly through the optical signal, which can improve the convenience and efficiency of the operation and improve the user experience.

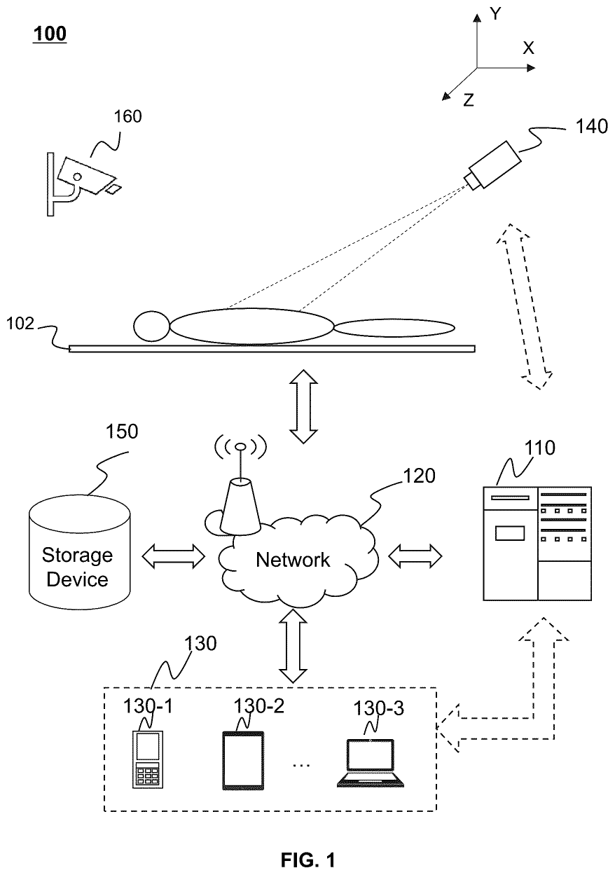

FIG. 1 is a schematic diagram illustrating an exemplary medical assistant system according to some embodiments of the present disclosure. As shown in FIG. 1, the medical assistant system 100 may include a processing device 110, a network 120, a terminal device 130, an optical projection device 140, a storage device 150, and an image acquisition device 160. In some embodiments, the optical projection device 140, the terminal device 130, the processing device 110, the storage device 150, and/or the image acquisition device 160 may be connected to and/or communicate with each other via a wireless connection, a wired connection, or a combination thereof. The connection among the components of the medical assistant system 100 may be variable. Merely by way of example, the optical projection device 140 may be connected to the processing device 110 through the network 120, as illustrated in FIG. 1. As another example, the optical projection device 140 may be connected to the processing device 110 directly. As a further example, the storage device 150 may be connected to the processing device 110 through the network 120, as illustrated in FIG. 1, or connected to the processing device 110 directly.

The processing device 110 may process data and/or information obtained from one or more components (e.g., the optical projection device 140, the terminal 130, the storage device 150, and/or the image acquisition device 160) of the medical assistant system 100. For example, the processing device 110 may obtain position information of a target (e.g., tumor) inside a subject (e.g., a patient) during an operation (e.g., a surgery operation). As another example, the processing device 110 may determine depth information of the target with respect to an operational region (e.g., a surgery region on a surface of the subject) of the subject based on the position information. As still another example, the processing device 110 may direct the optical projection device 140 to project an optical signal representing the depth information on the surface of the subject.

In some embodiments, the processing device 110 may be in communication with a computer-readable storage medium (e.g., the storage device 150) and may execute instructions stored in the computer-readable storage medium.

In some embodiments, the processing device 110 may be a single server or a server group. The server group may be centralized or distributed. In some embodiments, the processing device 110 may be local or remote. For example, the processing device 110 may access information and/or data stored in the optical projection device 140, the terminal device 130, and/or the storage device 150 via the network 120. As another example, the processing device 110 may be directly connected to the optical projection device 140, the terminal device 130, and/or the storage device 150 to access stored information and/or data. In some embodiments, the processing device 110 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the processing device 110 may be implemented by a computing device. For example, the computing device may include a processor, a storage, an input/output (I/O), and a communication port. The processor may execute computer instructions (e.g., program codes) and perform functions of the processing device 110 in accordance with the techniques described herein. The computer instructions may include, for example, routines, programs, objects, components, data structures, procedures, modules, and functions, which perform particular functions described herein. In some embodiments, the processing device 110, or a portion of the processing device 110 may be implemented by a portion of the terminal device 130.

In some embodiments, the processing device 110 may include multiple processing devices. Thus operations and/or method steps that are performed by one processing device as described in the present disclosure may also be jointly or separately performed by the multiple processing devices. For example, if in the present disclosure the, the medical assistant system 100 executes both operation A and operation B, it should be understood that operation A and operation B may also be performed by two or more different processing devices jointly or separately (e.g., a first processing device executes operation A and a second processing device executes operation B, or the first and second processing devices jointly execute operations A and B).

The network 120 may include any suitable network that can facilitate the exchange of information and/or data for the medical assistant system 100. In some embodiments, one or more components (e.g., the optical projection device 140, the terminal device 130, the processing device 110, the storage device 150, the image acquisition device 160) of the medical assistant system 100 may communicate information and/or data with one or more other components of the medical assistant system 100 via the network 120. In some embodiments, the network 120 may include one or more network access points. For example, the network 120 may include wired and/or wireless network access points such as base stations and/or internet exchange points through which one or more components of the medical assistant system 100 may be connected to the network 120 to exchange data and/or information.

The terminal device 130 may include a mobile device 130-1, a tablet computer 130-2, a laptop computer 130-3, or the like, or any combination thereof. In some embodiments, the mobile device 130-1 may include a smart home device, a wearable device, a mobile device, a virtual reality device, an augmented reality device, or the like, or any combination thereof. In some embodiments, the terminal device 130 may be part of the processing device 110.

The optical projection device 140 may be configured to project an optical signal on a surface of a subject. For example, as mentioned above, when an operation (e.g., a treatment operation) is performed on the subject, the optical projection device 140 may project an optical signal representing the depth information of the target (e.g., a tumor) with respect to an operational region (e.g., a surgery region on a surface of the subject) of the subject on the surface of the subject lying on a table 102. As another example, the optical projection device 140 may project the optical signal on the surface of the subject based on a projection instruction provided by the processing device 110. The projection instruction may be configured to direct a projection operation of the optical projection device 140 and may be associated with signal information included in the optical signal.

In some embodiments, the optical projection device 140 may include a liquid crystal display (LCP) projection device, a digital lighting process (DLP) projection device, a cathode ray tube (CRT) projection device, or the like, or any combination thereof.

In some embodiments, the optical projection device 140 may be disposed at various suitable positions, as long as the optical signal is projected on the surface of the subject based on the projection instruction. For example, the optical projection device 140 may be disposed on a component (e.g., a scanner, a table, a gantry) of a medical device (e.g., a medical imaging device, a radiotherapy device). As another example, the optical projection device 140 may be disposed on a wall of a room that is used to place the subject.

The storage device 150 may store data/information obtained from the processing device 110, the optical projection device 140, the terminal device 130, and/or any other component of the medical assistant system 100. In some embodiments, the storage device 150 may include a mass storage, a removable storage, a volatile read-and-write memory, a read-only memory (ROM), or the like, or any combination thereof. In some embodiments, the storage device 150 may store one or more programs and/or instructions to perform exemplary methods described in the present disclosure.

In some embodiments, the storage device 150 may be connected to the network 120 to communicate with one or more other components (e.g., e.g., the optical projection device 140, the terminal device 130, the processing device 110, the image acquisition device 160) of the medical assistant system 100. One or more components of the medical assistant system 100 may access the data or instructions stored in the storage device 150 via the network 120. In some embodiments, the storage device 150 may be directly connected to or communicate with one or more other components of the medical assistant system 100. In some embodiments, the storage device 150 may be part of the processing device 110.

The image acquisition device 160 (also referred to as a “first acquisition device”) may be configured to obtain image data (e.g., an optical image) of the subject. In some embodiments, the image data of the subject may be used to establish a subject model corresponding to the subject. In some embodiments, the image acquisition device 160 may obtain the image data of the subject before or during the operation. For example, the image acquisition device 160 may be directed to obtain the image data of the subject during the operation continuously or intermittently (e.g., periodically) so that the subject model corresponding to the subject may be updated in real-time or intermittently.

In some embodiments, the image acquisition device 160 may include a camera, an imaging sensor, or the like, or any combination thereof. Exemplary cameras may include a red-green-blue (RGB) camera, a depth camera, a time of flight (TOF) camera, a binocular camera, a structured illumination camera, a stereo triangulation camera, a sheet of light triangulation device, an interferometry device, a coded aperture device, a stereo matching device, or the like, or any combination thereof. Exemplary imaging sensors may include a radar sensor, a 3D laser imaging sensor, or the like, or any combination thereof.

In some embodiments, the image acquisition device 160 may be disposed at various suitable positions, as long as the subject is within a field of view (FOV) of the image acquisition device 160 for obtaining the image data of the subject. For example, the image acquisition device 160 may be disposed on a component of a medical device or a wall of the room that is used to place the subject.

In some embodiments, the medical assistant system 100 may include two or more projection devices and/or two or more image acquisition devices. In some embodiments, a count of the optical projection device(s) 140, a count of the image acquisition device(s) 160, the position of the optical projection device 140, and/or the position of the image acquisition device 160 may be set or adjusted according to different operational situations, which are not limited herein.

In some embodiments, the medical assistant system 100 may further include a medical imaging device (also referred to as a “second acquisition device”). The medical imaging device may be configured to obtain medical image data (e.g., a scanned image) of the subject. In some embodiments, the medical image data of the subject may be used to determine the position information of the target. Further, depth information of the target with respect to the operational region of the subject may be determined based on the position information. In some embodiments, the medical imaging device may obtain the medical image data of the subject before or during the operation.

In some embodiments, the medical imaging device may include a single modality imaging device. For example, the medical imaging device may include a computed tomography (CT) device, a magnetic resonance imaging (MRI) device, a positron emission tomography (PET) device, an X-ray imaging device, a single-photon emission computed tomography (SPECT) device, an ultrasound device, or the like. In some embodiments, the medical imaging device may include a multi-modality imaging device. Exemplary multi-modality imaging devices may include a positron emission tomography-computed tomography (PET-CT) device, a positron emission tomography-magnetic resonance imaging (PET-MRI) device, a computed tomography-magnetic resonance imaging (CT-MRI) device, or the like. The multi-modality scanner may perform multi-modality imaging simultaneously. For example, the PET-CT device may generate structural X-ray CT image data and functional PET image data simultaneously in a single scan. The PET-MRI device may generate MRI data and PET data simultaneously in a single scan.

It should be noted that the above description regarding the medical assistant system 100 is merely provided for the purposes of illustration, and is not intended to limit the scope of the present disclosure. For persons having ordinary skills in the art, multiple variations and modifications may be made under the teachings of the present disclosure. However, those variations and modifications do not depart from the scope of the present disclosure. In some embodiments, the medical assistant system 100 may include one or more additional components, and/or one or more components of the medical assistant system 100 described above may be omitted. In some embodiments, a component of the medical assistant system 100 may be implemented on two or more sub-components. Two or more components of the medical assistant system 100 may be integrated into a single component.

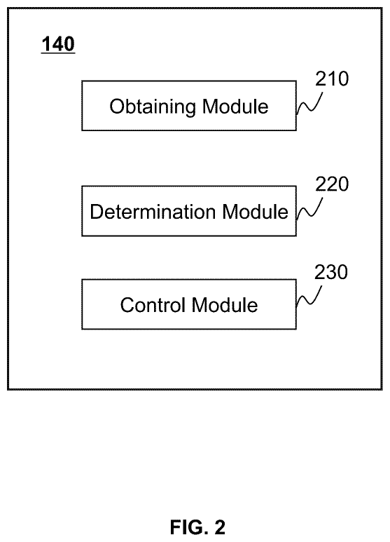

FIG. 2 is a block diagram illustrating an exemplary processing device according to some embodiments of the present disclosure. In some embodiments, the modules illustrated in FIG. 2 may be implemented on a computing device. In some embodiments, the processing device 110 may include an obtaining module 210, a determination module 220, and a control module 230.

The obtaining module 210 may be configured to obtain position information of a target inside a subject during an operation. The target may include a region of the subject that needs to be treated or diagnosed. The operation may refer to an invasive operation on the subject or the target for diagnosis or treatment. The position information of the target may include information regarding an absolute position of the target and/or information regarding a relative position of the target. In some embodiments, the information regarding the absolute position of the target may include coordinates of the target in a coordinate system. In some embodiments, the information regarding the relative position of the target may include a positional relationship between the target and a reference object (e.g., the subject (e.g., a surface of the subject), the table 102, the optical projection device 140, the image acquisition device 160). More descriptions regarding the obtaining of the position information of the target may be found elsewhere in the present disclosure. See, e.g., operation 302 and relevant descriptions thereof.

The determination module 220 may be configured to determine depth information of the target with respect to an operational region of the subject based on the position information of the target. The operational region may refer to an invasive region (e.g., a region on a surface of the subject) corresponding to the operation. The depth information of the target with respect to the operational region may refer to a positional relationship between the target and the operational region. In some embodiments, the depth information may include a distance between the target and the operational region, an angle between the target and the operational region, etc. In some embodiments, the determination module 220 may determine the depth information of the target with respect to the operational region of the subject based on the position information of the target. More descriptions regarding the determination of the depth information may be found elsewhere in the present disclosure. See, e.g., operation 304 and relevant descriptions thereof.

The control module 230 may be configured to direct an optical projection device (e.g., the optical projection device 140 illustrated in FIG. 1) to project an optical signal representing the depth information on a surface of the subject. The optical signal can provide reference information or assistant information for the operation to be performed or being performed on the target. In some embodiments, the control module 230 may determine a projection instruction based on the depth information of the target and direct the optical projection device to project the optical signal based on the projection instruction. In some embodiments, the projection instruction may be associated with the signal information included in the optical signal. In some embodiments, the projection instruction may be configured to direct a projection operation (e.g., an operation for projecting the optical signal) of the optical projection device. In some embodiments, the control module 230 may determine the projection instruction based on an input by a user (e.g., a doctor, a technician). In some embodiments, the control module 230 may automatically determine the projection instruction based on the depth information of the target with respect to the operational region of the subject. More descriptions regarding the control of the optical projection device may be found elsewhere in the present disclosure. See, e.g., operation 306 and relevant descriptions thereof.

The modules in the processing device 110 may be connected to or communicate with each other via a wired connection or a wireless connection. The wired connection may include a metal cable, an optical cable, a hybrid cable, or the like, or any combination thereof. The wireless connection may include a Local Area Network (LAN), a Wide Area Network (WAN), a Bluetooth, a ZigBee, a Near Field Communication (NFC), or the like, or any combination thereof.

It should be noted that the above descriptions of the processing device 110 are provided for the purposes of illustration, and not intended to limit the scope of the present disclosure. For persons having ordinary skills in the art, various variations and modifications may be conducted under the guidance of the present disclosure. However, those variations and modifications do not depart from the scope of the present disclosure. In some embodiments, the processing device 110 may include one or more other modules. For example, the processing device 110 may include a storage module used to store data generated by the modules in the processing device 110. In some embodiments, two or more of the modules may be combined as a single module, and any one of the modules may be divided into two or more units.

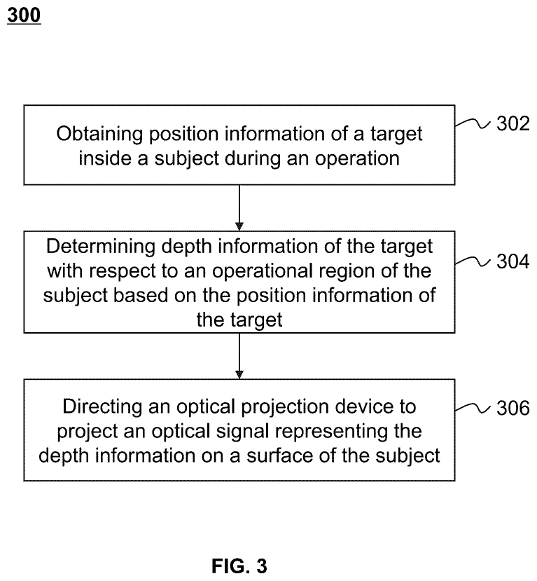

FIG. 3 is a flowchart illustrating an exemplary process for medical assistant according to some embodiments of the present disclosure. In some embodiments, process 300 may be executed by the medical assistant system 100. For example, the process 300 may be stored in the storage device 150 in the form of instructions (e.g., an application), and invoked and/or executed by the processing device 110. The operations of the illustrated process presented below are intended to be illustrative. In some embodiments, the process 300 may be accomplished with one or more additional operations not described, and/or without one or more of the operations discussed. Additionally, the order in which the operations of the process 300 as illustrated in FIG. 3 and described below is not intended to be limiting.

In 302, the processing device 110 (e.g., the obtaining module 210) may obtain position information of a target inside a subject during an operation.

The target may include a region of the subject that needs to be treated or diagnosed. For example, the target may include at least part of a malignant tissue (e.g., a tumor, a cancer-ridden organ, a non-cancerous target of radiation therapy). Merely by way of example, the target may be a lesion (e.g., a tumor, a lump of abnormal tissue), an organ with a lesion, a tissue with a lesion, or any combination thereof.

The operation may refer to an invasive operation on the subject or the target for diagnosis or treatment. Exemplary operations may include an exploratory operation, a therapeutic operation, a cosmetic operation, or the like, or any combination thereof. The exploratory operation may be performed to aid or confirm the diagnosis. The therapeutic operation may be performed to treat the target that is previously diagnosed. The cosmetic operation may be performed to subjectively improve the appearance of an otherwise normal structure.

The position information of the target may include information regarding an absolute position of the target and/or information regarding a relative position of the target. In some embodiments, the information regarding the absolute position of the target may include coordinates of the target in a coordinate system. For example, the information regarding the absolute position of the target may include a coordinate (e.g., a longitude, a latitude, and an altitude) of at least one point on the target under the world coordinate system. As another example, the processing device 110 may establish a three-dimensional (3D) coordinate system and the information regarding the absolute position of the target may include a coordinate of at least one point on the target under the 3D coordinate system. For instance, as shown in FIG. 1, a central point of an upper surface of the table 102 may be designated as an origin of the 3D coordinate system, a long side of the upper surface of the table 102 may be designated as an X axis of the 3D coordinate system, a short side of the upper surface of the table 102 may be designated as a Z axis of the 3D coordinate system, and a vertical direction of the table 102 may be designated as a Y axis of the 3D coordinate system. Accordingly, each point of the target can be represented by 3D coordinates corresponding to the 3D coordinate system. In some embodiments, the information regarding the relative position of the target may include a positional relationship between the target and a reference object (e.g., the subject (e.g., a surface of the subject), the table 102, the optical projection device 140, the image acquisition device 160).

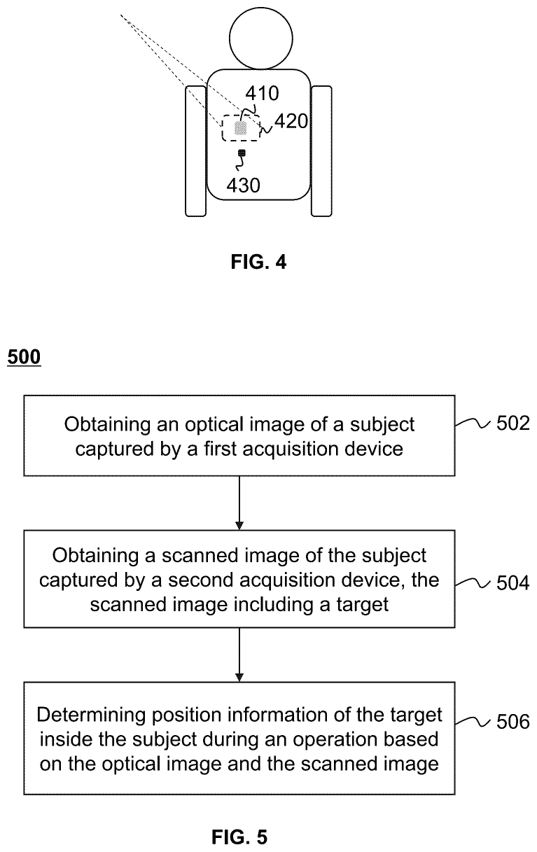

In some embodiments, the processing device 110 may obtain an optical image of the subject captured by a first acquisition device (e.g., the image acquisition device 160) and a scanned image (which includes the target) of the subject captured by a second acquisition device (e.g., the medical acquisition device). Further, the processing device 110 may determine the position information of the target inside the subject during the operation based on the optical image and the scanned image. More descriptions regarding the determination of the position information may be found elsewhere in the present disclosure (e.g., FIGS. 4 and 5, and the descriptions thereof).

In 304, the processing device 110 (e.g., the determination module 220) may determine depth information of the target with respect to an operational region of the subject based on the position information of the target.

The operational region may refer to an invasive region (e.g., a region on a surface of the subject) corresponding to the operation. For example, the operation may be performed on the target through the operational region. As another example, the operational region may be opened to expose the target, so that the operation may be performed on the target. In some embodiments, an area of the operational region may be different from or the same as an area of the target (e.g., an upper surface of the target, a cross region of the target). For example, when the operation is a minimally-invasive operation, the area of the operational region may be less than the area of the target. As another example, when the operation is an open operation, the area of the operational region may be equal to or larger than the area of the target.

In some embodiments, the operational region of the subject may be determined based on a system default setting (e.g., statistic information) or set manually by a user (e.g., a technician, a doctor, a physicist). For example, the processing device 110 may determine the operational region of the subject based on a treatment plan (e.g., a type of the operation, a type of the target, the position information of the target). As another example, a doctor may manually determine the operational region on the surface of the subject based on the treatment plan.

The depth information of the target with respect to the operational region may refer to a positional relationship between the target and the operational region. In some embodiments, the depth information may include a distance between the target and the operational region, an angle between the target and the operational region, etc.

In some embodiments, the distance between the target and the operational region may be a distance between a point (e.g., a surface point, a central point on a surface of the target, a central point, any interior point) of the target and a point (e.g., a boundary point, a central point, any interior point) of the operational region, a distance between a surface (e.g., an upper surface, a lower surface, a horizontal cross-sectional surface, a central horizontal cross-sectional surface, any cross-sectional surface) of the target and a surface (e.g., a horizontal surface level) of the operational region, a distance between a point of the target and a surface of the operational region, a distance between a surface of the target and a point of the operational region, etc.

In some embodiments, the angle between the target and the operational region may include an angle between a surface (e.g., the upper surface, the lower surface, the horizontal cross-sectional surface, the central horizontal cross-sectional surface, any cross-sectional surface) of the target and a surface (e.g., the horizontal surface level) of the operational region, an angle between a connecting line of a point (e.g., a surface point, a central point on a surface of the target, a central point, any interior point) of the target and a point (e.g., a boundary point, a central point, any interior point) of the operational region and a vertical direction (e.g., Y direction illustrated in FIG. 1) or a horizontal direction (e.g., X direction illustrated in FIG. 1) of the table 102, etc.

Merely for illustration, the distance between the target and the operational region may be the distance between the upper surface of the target and the horizontal surface level of the operational region, and the angle between the target and the operational region may be the angle between the upper surface of the target and the horizontal surface level of the operational region.

In some embodiments, the processing device 110 may determine the depth information of the target with respect to the operational region of the subject based on the position information of the target. For example, if the position information of the target includes the information regarding the absolute position of the target and point(s) of the target and point(s) of the operational region are represented by coordinates, the depth information may be determined based on the coordinates of the point(s) of the target and the point(s) of the operational region. For instance, if a coordinate of a central point of the target is (X₁, Y₁, Z₁) and a coordinate of a central point of the operational region is (X₂, Y₂, Z₂), the depth information may be represented a vector (X₁-X₂, Y₁-Y₂, Z₁-Z₂). As another example, if the position information of the target includes the information regarding the relative position of the target and the reference object is the operational region, the processing device 110 may directly designate the relative position information as the depth information. As still another example, if the position information of the target includes the information regarding the relative position of the target and the reference object is another object (e.g., a portion of the subject other than the operational region, the table 102, the optical projection device 140, the image acquisition device 160) instead of the operational region, the processing device 110 may obtain relative position information between the reference object and the operational region, and then determine the depth information of the target based on the position information of the target and the relative position information between the operational region and the reference object.

In 306, the processing device 110 (e.g., the control module 230) may direct an optical projection device (e.g., the optical projection device 140 illustrated in FIG. 1) to project an optical signal representing the depth information on a surface of the subject.

The optical signal can provide reference information or assistant information for the operation to be performed or being performed on the target. In some embodiments, signal information included in the optical signal may include color information of the optical signal, position information of the optical signal projected on the surface of the subject, or the like, or any combination thereof.

In some embodiments, the color information of the optical signal may indicate the depth information of the target with respect to the operational region (or a surface level of the operational region) of the subject. For example, different colors may correspond to different depths between the target and the operational region of the subject. For instance, a blue light may indicate that a distance between the target and the operational region exceeds 2 centimeters, a green light may indicate that the distance between the target and the operational region of the subject is within 2 centimeters, a yellow light may indicate that the target has been operated, etc. As another example, different saturations and/or different brightness may correspond to different depths. For instance, a distance between the target and the operational region of the subject corresponding to a low saturation and/or a low brightness of the optical signal may be larger than a distance between the target and the operational region of the subject corresponding to a high saturation and/or a high brightness of the optical signal.

In some embodiments, the color information of the optical signal may be associated with a type of the target. In some embodiments, different types of targets may correspond to different color information of optical signals. For example, if the target is an organ with a lesion, a color of an optical signal corresponding to the organ may be more conspicuous than a color of an optical signal corresponding to the lesion.

In some embodiments, the position information of the optical signal projected on the surface of the subject may include relevant information (e.g., shape, boundary, coordinate, size) associated with a projection point or a projection region where the optical signal is projected.

In some embodiments, the signal information included in the optical signal may also include or indicate other reference information or assistant information associated with the operation. In some embodiments, the signal information may include or indicate age information, gender information, prompt information, or the like, or any combination thereof. For example, a color of an optical signal corresponding to the child or the aged may be more conspicuous than a color of an optical signal corresponding to the adult. As another example, when an abnormal condition (e.g., bleeding occurs in an organ at risk (OAR), an operational instrument is left over) occurs, a red light may be projected on a region around the operational region to prompt the user to stop the operation.

In some embodiments, the optical signal may include a plurality of portions indicating various reference information or assistant information. For example, as illustrated in FIG. 4, the optical signal may include a first portion 410 for indicating a target of a liver region, a second portion 420 for indicating an OAR of the liver region, and a third portion 430 for indicating a region other than the liver region. Further, the first portion 410 of the optical signal may be a green light, the second portion 420 of the optical signal may be a yellow light, and the third portion 430 of the optical signal may be a blue light. Accordingly, the user can distinguish the target from the liver region (or the OAR) through the optical signal. As another example, the first portion 410 of the optical signal may be used to distinguish the target, the second portion 420 of the optical signal may be used to indicate the depth information of the target, and the third portion 430 of the optical signal may provide other reference information or assistant information (e.g., age information, prompt information).

In some embodiments, the processing device 110 may determine a projection instruction based on the depth information of the target and direct the optical projection device to project the optical signal based on the projection instruction. In some embodiments, the projection instruction may be associated with the signal information included in the optical signal. In some embodiments, the projection instruction may be configured to direct a projection operation (e.g., an operation for projecting the optical signal) of the optical projection device. Exemplary projection operations may include controlling the projection device to move a projection position, controlling the projection device to move a projection angle, controlling the projection device to select a projection color of the optical signal, controlling the projection device to alter the projection color of the optical signal, controlling the projection device to project, controlling the projection device to stop projecting, or the like, or any combination thereof. In some embodiments, the projection instruction may include parameter(s) (e.g., the projection angle, the projection position, the projection region, the projection color) of the projection device, parameter(s) (e.g., a projection period) of the projection operation, etc.

In some embodiments, the processing device 110 may determine the projection instruction based on an input by a user (e.g., a doctor, a technician). For example, the user may determine the signal information associated with the optical signal based on the depth information of the target with respect to the operational region of the subject, and input a projection instruction including the signal information associated with the optical signal.

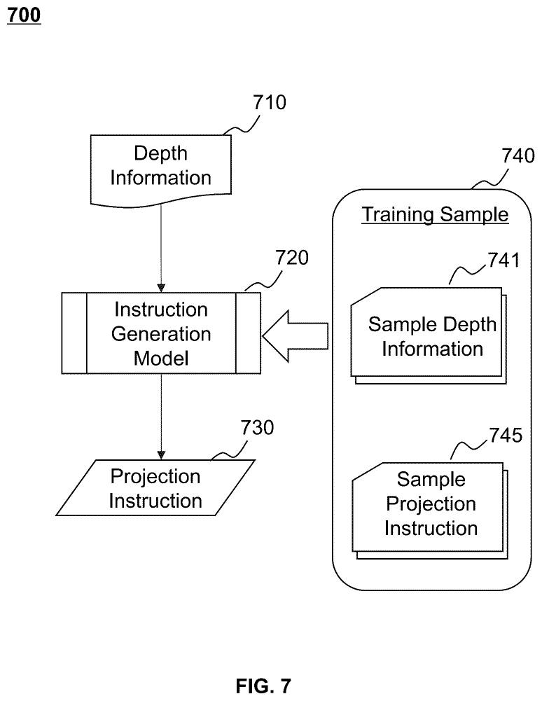

In some embodiments, the processing device 110 may automatically determine the projection instruction based on the depth information of the target with respect to the operational region of the subject. For example, the processing device 110 may obtain an instruction generation model and determine the projection instruction based on the depth information of the target with respect to the operational region of the subject and the instruction generation model. More descriptions regarding the determination of the projection instruction may be found elsewhere in the present disclosure (e.g., FIG. 7 and the descriptions thereof).

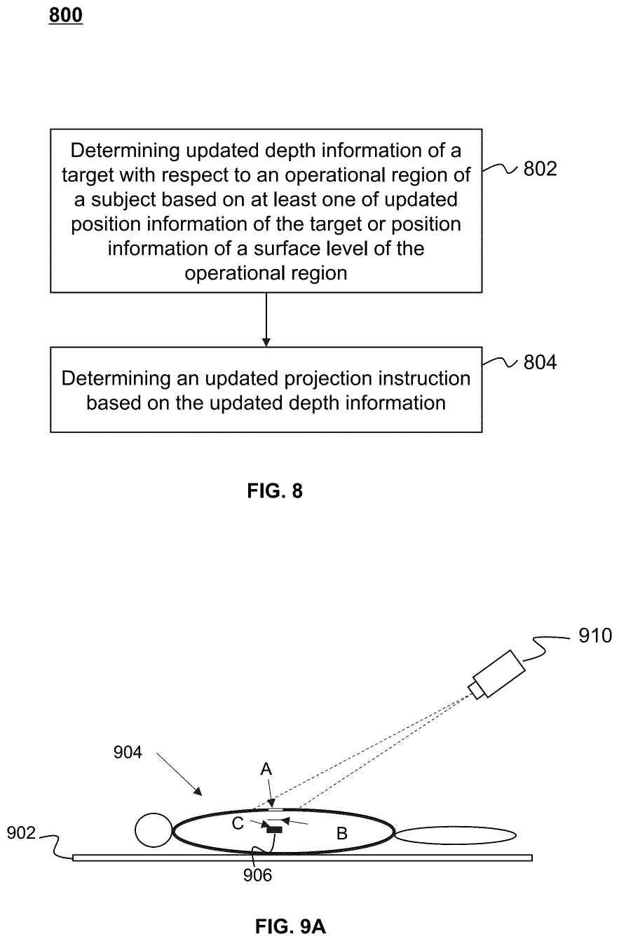

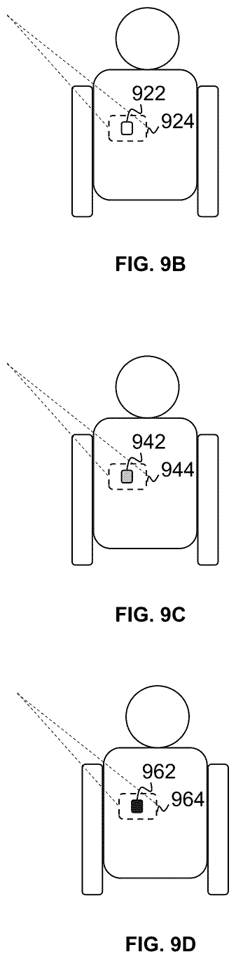

In some embodiments, during the operation performed on the subject, the position information of the target and/or position information of a surface level of the operational region of the subject may change. The depth information of the target with respect to the operational region of the subject may change accordingly. In order to provide accurate reference information or assistant information, the processing device 110 may determine updated depth information of the target with respect to the operational region of the subject based on at least one of updated position information of the target or the position information of the surface level of the operational region, and determine an updated projection instruction based on the updated depth information. More descriptions regarding the determination of the updated projection instruction may be found elsewhere in the present disclosure (e.g., FIGS. 8-9D and the descriptions thereof).

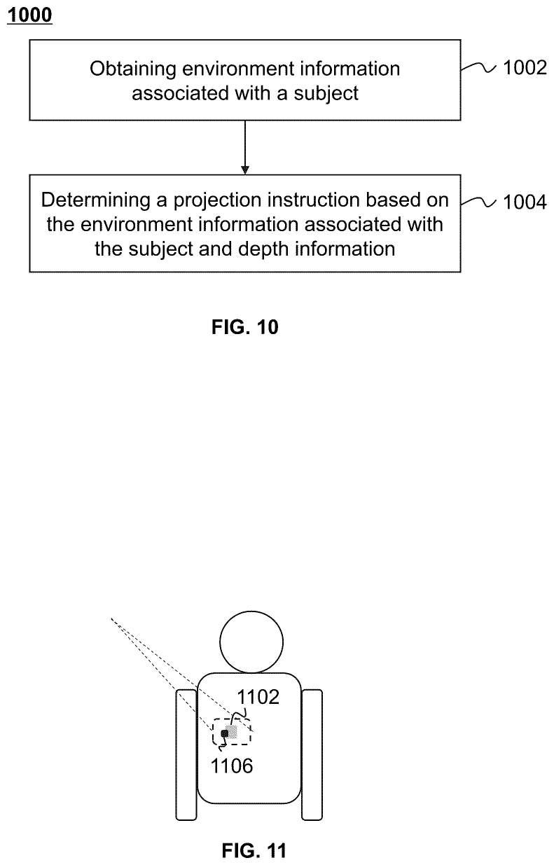

In some embodiments, the processing device 110 may obtain environment information (e.g., position information of a user (or a portion of the user), position information of an operational instrument, environmental brightness information, object(s) that may affect the projection of the optical signal) associated with the subject, and then determine the projection instruction based on the environment information associated with the subject and the depth information of the target. More descriptions regarding the determination of the projection instruction may be found elsewhere in the present disclosure (e.g., FIGS. 10-11B and the descriptions thereof).

According to some embodiments of the present disclosure, the depth information of the target with respect to the operational region of the subject may be determined, and the optical signal representing the depth information may be projected on the surface of the subject. Accordingly, the user can obtain the depth information directly through the optical signal, which can improve the convenience and efficiency of the operation and improve the user experience.

It should be noted that the description of the process 300 is provided for the purposes of illustration, and is not intended to limit the scope of the present disclosure. For persons having ordinary skills in the art, various variations and modifications may be conducted under the teaching of the present disclosure. For example, when the optical signal is projected, the processing device 110 may display the optical signal on a user interface, and the user can check and/or obtain the signal information on the user interface. However, those variations and modifications may not depart from the protection of the present disclosure.

FIG. 5 is a flowchart illustrating an exemplary process for determining position information of a target inside a subject during an operation according to some embodiments of the present disclosure. In some embodiments, the process 500 may be performed to achieve at least part of operation 302 as described in connection with FIG. 3.

In 502, the processing device 110 (e.g., the obtaining module 210) may obtain an optical image of a subject captured by a first acquisition device (e.g., the image acquisition device 160).

In some embodiments, the processing device 110 may obtain the optical image from the first acquisition device or a storage device (e.g., the storage device 150, a database, or an external storage device) that stores the optical image of the subject.

In 504, the processing device 110 (e.g., the obtaining module 210) may obtain a scanned image of the subject captured by a second acquisition device.

In some embodiments, the scanned image may include the target. In some embodiments, the scanned image of the subject may include a medical image including structural information of the subject. Exemplary scanned images may include a CT image, an MR image, a PET image, an X-ray image, an ultrasound image, or the like. In some embodiments, the scanned image may be a 3-dimensional image including a plurality of slices.

In some embodiments, the processing device 110 may obtain the scanned image from the second acquisition device (e.g., a medical imaging device) or a storage device (e.g., the storage device 150, a database, or an external storage device) that stores the scanned image of the subject.

In 506, the processing device 110 (e.g., the obtaining module 210) may determine position information of the target inside the subject during an operation based on the optical image and the scanned image.

In some embodiments, the processing device 110 may establish a subject model corresponding to the subject based on the optical image. The subject model may represent an external contour of the subject. Exemplary subject models may include a mesh model (e.g., a surface mesh model, a human mesh model), a 3D mask, a kinematic model, or the like, or any combination thereof. For example, the processing device 110 may establish the human mesh model corresponding to the subject based on the optical image according to a technique disclosed in U.S. patent application Ser. No. 16/863,382, which is incorporated herein by reference.

In some embodiments, the processing device 110 may align the scanned image with the subject model. In some embodiments, the processing device 110 may align the scanned image with the subject model based on a calibration technique (e.g., a calibration matrix). The calibration matrix may refer to a transfer matrix that converts a first coordinate system corresponding to the subject model and a second coordinate system corresponding to the scanned image to a same coordinate system. For example, the calibration matrix may be configured to convert the first coordinate system corresponding to the subject model to the second coordinate system corresponding to the scanned image. As another example, the calibration matrix may be configured to convert the second coordinate system corresponding to the scanned image to the first coordinate system corresponding to the subject model. As still another example, the calibration matrix may be configured to convert the second coordinate system corresponding to the scanned image and the first coordinate system corresponding to the subject model to a reference coordinate system.

In some embodiments, the processing device 110 may align the scanned image with the subject model based on a registration algorithm. Exemplary registration algorithms may include an AI-based registration algorithm, a grayscale information-based registration algorithm, a transform domain-based registration algorithm, a feature-based registration algorithm, or the like, or any combination thereof.

In some embodiments, the processing device 110 may determine the position information of the target inside the subject during the operation based on the aligned scanned image and the aligned subject model. For example, after the scanned image is aligned with the subject model, the scanned image and the subject model may be converted to the same coordinate system. Accordingly, point(s) of the target and point(s) of the subject may be represented by corresponding coordinates in the same coordinate system. The processing device 110 may determine the position information of the target inside the subject during the operation based on the coordinates under the same coordinate system.

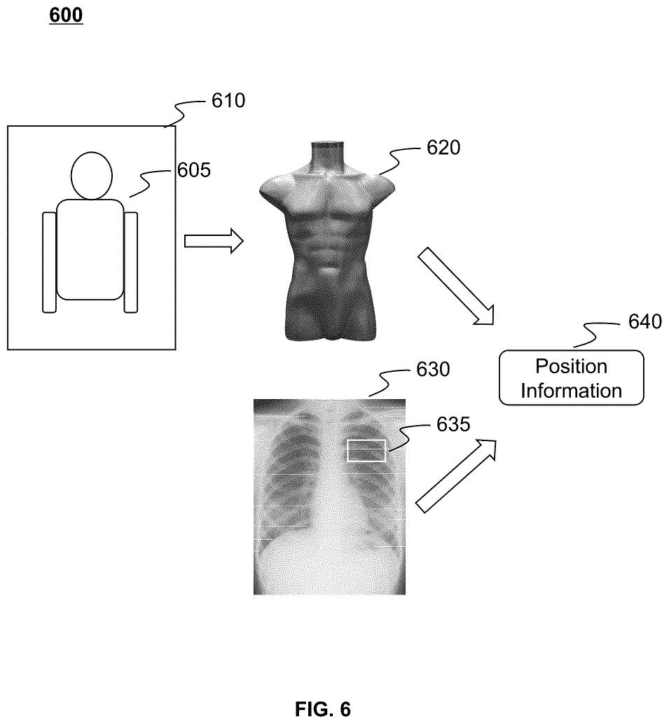

Merely by way of example, as illustrated in FIG. 6, an optical image 610 of a subject 605 may be obtained, and a subject model 620 corresponding to the subject 605 may be established based on the optical image 610. A scanned image 630 of the subject 605 may be obtained, wherein the scanned image 630 may include a target 635. Further, the scanned image 630 may be aligned with the subject model 620. Correspondingly, position information 640 of the target 635 inside the subject 605 during an operation may be determined based on the aligned subject model and the aligned scanned image.

In some embodiments, the processing device 110 may align the scanned image with the optical image, and determine the position information of the target inside the subject during the operation based on an aligned image. For example, the processing device 110 may input the scanned image and the optical image into an image registration model, and the image registration model may output the aligned image. The processing device 110 may determine the position information of the target inside the subject during the operation based on the aligned image. The image registration model may be obtained by training an initial image registration model (e.g., an initial deep learning model) based on a plurality of training samples. Each of the plurality of training samples may include a sample optical image and a sample scanned image of a sample subject as an input of the initial image registration model, and a sample aligned image of the sample subject as a label. In some embodiments, the plurality of training samples may include historical image data.

It should be noted that the description of the process 500 is provided for the purposes of illustration, and is not intended to limit the scope of the present disclosure. For persons having ordinary skills in the art, various variations and modifications may be conducted under the teaching of the present disclosure. However, those variations and modifications may not depart from the protection of the present disclosure.

FIG. 7 is a schematic diagram illustrating an exemplary process for determining a projection instruction according to some embodiments of the present disclosure.

As shown in FIG. 7, in some embodiments, depth information 710 of a target may be input into an instruction generation model 720, and the instruction generation model 720 may output a projection instruction 730.

In some embodiments, the instruction generation model 720 may include a convolutional neural network (CNN), a deep neural network (DNN), a recurrent neural network (RNN), or the like, or any combination thereof.

In some embodiments, the instruction generation model 720 may be obtained by training an initial instruction generation model based on a plurality of training samples 740. In some embodiments, each of the plurality of training samples 740 may include sample depth information 741 of a sample target inside a sample subject as an input of the initial image prediction model, and a sample projection instruction 745 as a label.

The obtaining of the sample depth information 741 may be similar to the obtaining of the depth information described in operations 502-506. The sample projection instruction 745 may be obtained based on a system default setting (e.g., statistic information) or set manually by a user (e.g., a technician, a doctor, a physicist). In some embodiments, the processing device 110 may obtain the plurality of training samples by retrieving (e.g., through a data interface) a database or a storage device.

During the training of the initial instruction generation model, the plurality of training samples may be input to the initial instruction generation model, and parameter(s) of the initial instruction generation model may be updated through one or more iterations. For example, the processing device 110 may input the sample depth information 741 of each training sample into the initial instruction generation model, and obtain a prediction result. The processing device 110 may determine a loss function based on the prediction result and the label (i.e., the corresponding sample projection instruction 745) of each training sample. The loss function may be associated with a difference between the prediction result and the label. The processing device 110 may adjust the parameter(s) of the initial instruction generation model based on the loss function to reduce the difference between the prediction result and the label, for example, by continuously adjusting the parameter(s) of the initial instruction generation model to reduce or minimize the loss function.

In some embodiments, the loss function may be a perceptual loss function, a squared loss function, a logistic regression loss function, etc.

In some embodiments, the instruction generation model may also be obtained according to other training manners. For example, the instruction generation model may be obtained based on an initial learning rate (e.g., 0.1) and/or an attenuation strategy using the plurality of training samples.

FIG. 8 is a flowchart illustrating an exemplary process for determining an updated projection instruction according to some embodiments of the present disclosure. In some embodiments, the process 800 may be performed to achieve at least part of operation 306 as described in connection with FIG. 3.

In 802, the processing device 110 (e.g., the determination module 220) may determine updated depth information of the target with respect to the operational region of the subject based on at least one of updated position information of the target or position information of a surface level of the operational region.

As described in connection with operation 306, during the operation performed on the subject, the position information of the target and/or the position information of a surface level of the operational region of the subject may change. For example, the position information of the target may change due to a slight movement of the subject. As another example, during the operation, one or more operation instruments (e.g., a grasper, a clamp, a surgical scissor) may be in contact with the operational region and a surface level of the operational region may change during the operation. Merely by way of example, as illustrated in FIG. 9A, a subject 904 may be lying on a table 902 and a target 906 is located inside the subject 904. An optical projection device 910 may be directed to project an optical signal on a surface of the subject 904. Before the operation is performed or the operation is to be performed, the surface level of the operational region may be represented by “A;” during the operation (e.g., the operation instrument gradually goes down and enters into the subject), the surface level of the operational region may be represented by “B” and “C.”

In some embodiments, the processing device 110 may obtain the position information of the surface level of the operational region based on the optical image of the subject captured by the first acquisition device. For example, as described in connection with FIG. 5 and FIG. 6, the processing device 110 may the position information of the surface level of the operational region based on the subject model established based on the optical image. As another example, the processing device 110 may obtain an updated optical image of the subject during the operation and establish an updated subject model corresponding to the subject based on the updated optical image. Further, the processing device 110 may align the scanned image with the updated subject model and determine the position information of the surface level of the operational region based on the aligned updated subject model.

In some embodiments, the determination of the updated depth information may be similar to the determination of the depth information described in operation 304 and FIG. 5, which is not repeated here.

In 804, the processing device 110 (e.g., the determination module 220) may determine an updated projection instruction based on the updated depth information of the target.

The determination of the updated projection instruction may be similar to the determination of the projection instruction described in operation 306. For example, the processing device 110 may obtain the instruction generation model as described in process 700, and determine the updated projection instruction based on the updated depth information.

In some embodiments, the updated projection instruction may be determined by updating parameter(s) in a previous projection instruction based on the updated depth information. For example, color information of the optical signal may be updated based on the updated depth information, and the updated projection instruction may be determined by adjusting a projection color based on the updated color information of the optical signal.

Merely by way of example, as illustrated in FIGS. 9B-9D, the projection of the optical signal in FIG. 9B corresponds to a situation that the surface level of the operational region is “A” illustrated in FIG. 9A, the projection of the optical signal in FIG. 9C corresponds to a situation that the surface level of the operational region is “B” illustrated in FIG. 9A, and the projection of the optical signal in FIG. 9D corresponds to a situation that the surface level of the operational region is “C” illustrated in FIG. 9A, wherein 922, 942, and 962 refer to signal portions indicating the target of a liver region and 924, 944, and 964 refer to signal portions indicating the OAR of the liver region. It can be seen that with the change (getting closer to the target) of the surface level of the operational region, the color of the signal portion indicating the target becomes darker.

In some embodiments, the processing device 110 may determine a motion amplitude of the surface level of the operational region and determine an estimated updating time for determining the updated projection instruction based on the motion amplitude.

In some embodiments, the motion amplitude of the surface level of the operational region may be determined based on a current surface level and one or more previous surface levels. For example, the first acquisition device may be directed to obtain optical images of the subject during the operation continuously or intermittently (e.g., periodically), and the processing device 110 may determine a motion amplitude of the surface level of the operational region between two or more adjacent optical images.

In some embodiments, the estimated updating time may indicate a time period that is required for determining the updated depth information of the target and accordingly determining the updated projection instruction, for example, as described in connection with operation 304 and FIG. 5, a required time period for aligning the subject model corresponding to the subject with the scanned image and further determining the position information of the target based on the aligned subject model and still further determining the depth information of the target based on the position information of the target.

In some embodiments, the larger the motion amplitude of the surface level of the operational region is, the larger the estimated updating time may be. In some embodiments, a relationship between the motion amplitude and the estimated updating time may be determined based on historical data.

In some embodiments, the processing device 110 may check the estimated updating time. For example, the processing device 110 may designate the estimated updating time determined based on the motion amplitude as a preliminary estimated updating time, and determine whether a confidence of the preliminary estimated updating time satisfies a condition. The confidence of the preliminary estimated updating time may be used to determine whether the estimated updating time is credible. In some embodiments, the confidence may be represented by a percentage, a grade, etc. For example, the confidence may be a value within a range from 0 to 1. In some embodiments, the confidence of the preliminary estimated updating time may be determined according to a confidence algorithm, which is not limited herein. The condition may be that the confidence of the preliminary estimated updating time is within a confidence range. The confidence range may be determined based on system default setting or set manually by the user, such as, a range from 0.5 to 0.8. If the confidence of the preliminary estimated updating time satisfies the condition, the processing device 110 may determine the preliminary estimated updating time as the estimated updating time. If the confidence of the preliminary estimated updating time doesn't satisfy the condition, the processing device 110 may update the preliminary estimated updating time.

In some embodiments, during the determination of the updated projection instruction, the processing device 110 may provide a prompt to inform that the optical signal is being updated. In some embodiments, the prompt may include an instruction used to direct the optical projection device to stop projecting the optical signal, an instruction used to direct the optical projection device to project another optical signal (e.g., an optical signal with a different color) indicating system update, a notification (e.g., a notification displayed on a user interface, a notification directly projected on the surface of the subject) indicating the estimated updating time, etc.

It should be noted that the description of the process 800 is provided for the purposes of illustration, and is not intended to limit the scope of the present disclosure. For persons having ordinary skills in the art, various variations and modifications may be conducted under the teaching of the present disclosure. However, those variations and modifications may not depart from the protection of the present disclosure.

FIG. 10 is a flowchart illustrating an exemplary process for determining a projection instruction according to some embodiments of the present disclosure. In some embodiments, the process 1000 may be performed to achieve at least part of operation 306 as described in connection with FIG. 3.

In 1002, the processing device 110 (e.g., the determination module 220) may obtain environment information associated with the subject.

The environment information associated with the subject may refer to information that may affect the operation performed on the target. For example, the environment information may include position information of a user (or a portion of the user), position information of an operational instrument, environmental brightness information, object(s) that may affect the projection of the optical signal, etc.

In some embodiments, the processing device 110 may obtain the environment information associated with the subject based on an optical image of the subject. For example, the processing device 110 may recognize object(s) from the optical image and determine whether the object(s) may affect the operation. If the object(s) may affect the operation (e.g., located in a projection region of an optical signal or block the projection of the optical signal), the object(s) may be determined as the environment information associated with the subject.

In 1004, the processing device 110 (e.g., the determination module 220) may determine a projection instruction based on the environment information associated with the subject and the depth information of the target.

In some embodiments, the processing device 110 may determine a preliminary projection instruction based on the depth information of the target (e.g., according to operation 306) and adjust parameter(s) in the preliminary projection instruction based on the environment information associated with the subject. For example, the processing device 110 may adjust a projection angle, so that the object(s) would not be in the projection region of the optical signal or would not block the projection of the optical signal. For example, as shown in FIG. 11, 1102 refers to an optical signal (or a signal portion) indicating the target. It can be seen that an object blocks a portion of the optical signal and a projection region 1106 corresponding to the object covers a portion of a projection region of the optical signal (or signal portion). Accordingly, in this situation, a projection instruction may be determined to adjust a projection angle of the optical signal (or signal portion).

It should be noted that the description of the process 1000 is provided for the purposes of illustration, and is not intended to limit the scope of the present disclosure. For persons having ordinary skills in the art, various variations and modifications may be conducted under the teaching of the present disclosure. However, those variations and modifications may not depart from the protection of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A method, implemented on a computing device having at least one processor and at least one storage device, the method comprising:
obtaining position information of a target inside a subject during an operation;
determining depth information of the target with respect to an operational region of the subject based on the position information; and
directing an optical projection device to project an optical signal representing the depth information on a surface of the subject.

2. The method of claim 1, wherein the obtaining position information of a target inside a subject during an operation includes:
obtaining an optical image of the subject captured by a first acquisition device;
obtaining a scanned image of the subject captured by a second acquisition device, the scanned image including the target; and
determining the position information of the target inside the subject during the operation based on the optical image and the scanned image.

3. The method of claim 2, wherein the determining the position information of the target inside the subject during the operation based on the optical image and the scanned image includes:
establishing a subject model corresponding to the subject based on the optical image;
aligning the scanned image with the subject model; and
determining the position information of the target inside the subject during the operation based on the aligned scanned image and the aligned subject model.

4. The method of claim 1, wherein the directing an optical projection device to project an optical signal representing the depth information on a surface of the subject includes:
determining a projection instruction based on the depth information, the projection instruction being configured to direct a projection operation of the optical projection device; and
directing the optical projection device to project the optical signal representing the depth information on the surface of the subject based on the projection instruction.

5. The method of claim 4, wherein the determining a projection instruction based on the depth information includes:
obtaining an instruction generation model; and
determining the projection instruction based on the depth information and the instruction generation model.

6. The method of claim 4, wherein the determining a projection instruction based on the depth information includes:
determining updated depth information of the target with respect to the operational region of the subject based on at least one of updated position information of the target or position information of a surface level of the operational region; and
determining an updated projection instruction based on the updated depth information.

7. The method of claim 4, wherein the determining a projection instruction based on the depth information includes:
obtaining environment information associated with the subject; and
determining the projection instruction based on the environment information associated with the subject and the depth information.

8. The method of claim 4, wherein the projection instruction is associated with signal information included in the optical signal, the signal information included in the optical signal including at least one of color information of the optical signal or position information of the optical signal projected on the surface of the subject.

9. The method of claim 8, wherein the color information of the optical signal indicates the depth information of the target with respect to the operational region.

10. The method of claim 8, wherein the color information of the optical signal is associated with a type of the target.

11. A system, comprising:
a controller configured to:
obtain position information of a target inside a subject during an operation, and
determine depth information of the target with respect of an operational region of the subject based on the position information, and
direct an optical projection device to project an optical signal representing the depth information on a surface of the subject; and
the optical projection device configured to project the optical signal representing the depth information on the surface of the subject.

12. The system of claim 11, wherein the obtaining position information of a target inside a subject during an operation includes:
obtaining an optical image of the subject captured by a first acquisition device;
obtaining a scanned image of the subject captured by a second acquisition device, the scanned image including the target; and
determining the position information of the target inside the subject during the operation based on the optical image and the scanned image.

13. The system of claim 12, wherein the determining the position information of the target inside the subject during the operation based on the optical image and the scanned image includes:
establishing a subject model corresponding to the subject based on the optical image;
aligning the scanned image with the subject model; and
determining the position information of the target inside the subject during the operation based on the aligned scanned image and the aligned subject model.

14. The system of claim 11, wherein the directing an optical projection device to project an optical signal representing the depth information on a surface of the subject includes:
determining a projection instruction based on the depth information, the projection instruction being configured to direct a projection operation of the optical projection device; and
directing the optical projection device to project the optical signal representing the depth information on the surface of the subject based on the projection instruction.

15. The system of claim 14, wherein the determining a projection instruction based on the depth information includes:
obtaining an instruction generation model; and
determining the projection instruction based on the depth information and the instruction generation model.

16. The system of claim 14, wherein the determining a projection instruction based on the depth information includes:
determining updated depth information of the target with respect to the operational region of the subject based on at least one of updated position information of the target or position information of a surface level of the operational region; and
determining an updated projection instruction based on the updated depth information.

17. The system of claim 14, wherein the determining a projection instruction based on the depth information includes:
obtaining environment information associated with the subject; and
determining the projection instruction based on the environment information associated with the subject and the depth information.

18. The system of claim 14, wherein the projection instruction is associated with signal information included in the optical signal, the signal information included in the optical signal including at least one of color information of the optical signal or position information of the optical signal projected on the surface of the subject.

19. The system of claim 18, wherein the color information of the optical signal indicates the depth information of the target with respect to the operational region.

20. A non-transitory computer readable medium, comprising executable instructions that, when executed by at least one processor, direct the at least one processor to perform a method, the method comprising:
obtaining position information of a target inside a subject during an operation;
determining depth information of the target with respect to an operational region of the subject based on the position information; and
directing an optical projection device to project an optical signal representing the depth information on a surface of the subject.
